(19) United States
(12) Patent Application Publication
(10) Pub. No.: US 2008/0140185 A1
(43) Pub. Date: Jun. 12, 2008

(54) Biodegradable and Biocompatible Peg-Based Poly(Ester-Urethanes)

(75) Inventors: Patrick F. Kiser, Salt Lake City, UT (US); Patrick Tresco, Sandy, UT (US)

Correspondence Address: THE MCCALLUM LAW FIRM, P. C., 685 BRIGGS STREET, PO BOX 929, ERIE, CO, 80516, US

(73) Assignee: UNIVERSITY OF UTAH RESEARCH FOUNDATION

(21) Appl. No.: 11/578,321

(22) PCT Filed: Apr. 15, 2005
(86) PCT No.: PCT/US2005/012879

§ 371 Date: Aug. 30, 2007

Related U.S. Application Data
(60) Provisional application No. 60/562,624, filed on Apr. 15, 2004.

(51) Int. Cl. A61F 2/82; C08G 18/42; A61K 47/30

(52) U.S. Cl. 623/1.42; 528/69; 514/772.3

(57) ABSTRACT

The compositions disclosed herein include biodegradable elastomers that provide a material for. medical purposes. They may further provide a drug delivery system for the treatment of a wide variety of medical conditions. In particular, the compositions provide biodegradable elastomers composed of a pre-polymeric block, a degradable sequence and a bis-functional linker. The usefulness of the present invention is that it provides a biodegradable elastomer for use with medical devices.

### CROSS REFERENCE TO RELATED APPLICATIONS

This application claims priority from U.S. Provisional Patent Application No. 60/562,624 filed Apr. 15, 2004.

### FIELD OF THE INVENTION

The present invention provides materials and methods for the production of biodegradable elastomers for medical use. The invention may further provide a delivery method for biologically active agents.

### BACKGROUND OF THE INVENTION

Many medical devices have been produced from poly(urethanes) due to their ability to have a wide range of physical properties. For instance, their form can range from a hard and brittle material to a soft and tacky one. Unfortunately, the ability to biodegrade is not an inherent characteristic. The ability to control the degradation rate of poly(urethanes) in-vivo would be a great improvement and greatly facilitate an increase in their use in the medical field.

### SUMMARY OF THE INVENTION

The present invention is directed to biodegradable elastomers of poly(ester-urethane) and to medical devices and compositions containing such biodegradable elastomers. The construction of the material is unique in that biodegradable ester linkages have been introduced to provide a biodegradable elastomer susceptible to degradation upon contact with physiological conditions. This is an improvement over traditional poly(urethanes) that are not susceptible to biodegradation. The biodegradable elastomers of the present invention can be produced by any method. In a particular embodiment, they are produced by reacting diisocyanates and polyols with cyclic lactones of the α-ahydroxyester. In another particular embodiment, they are composed of the reaction product of poly-ethylene glycol-b1-α-hydroxyacids condensed with bis-isocyanates and short chain diols. Biodegradation of the elastomer results in products biocompatible with a patient, such as lactic acid, glycolic acid, amines, alcohols, carbon dioxide and the like. The degradation rate may be manipulated by varying the properties in the α-hydroxyacids blocks themselves, such as molecular weight, chemical structure and the like. One skilled in the art will understand how to create the desired biodegradable elastomers of the present invention with the appropriate degradation rate by utilizing the inherent properties of the base components.

### DETAILED DESCRIPTION OF THE INVENTION

The present invention may be understood more readily by reference to the following detailed description of particular embodiments of the invention and Examples included therein.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present invention and/or methods are disclosed and described, it is to be understood that this invention is not limited to specific reagents or synthetic procedures, as such may, of course, vary, unless it is otherwise indicated. It is also to be understood that the terminology used herein is for the purpose of describing particular embodiments only and is not intended to be limiting.

### FIGURES

The following drawings form part of the present specification and are included to further demonstrate certain embodiments. These embodiments may be better understood by reference to one or more of these drawings in combination with the detailed description of specific embodiments presented herein.

FIG. 1 illustrates an exemplary biodegradable elastomer of the present invention in a linear degradable block co-polymer structure according to an embodiment of the present invention. A represents the pre-polymer block, B the end group functionality, C the degradable sequence, D the bis-functionalized linker component and X is the degree of polymerization.

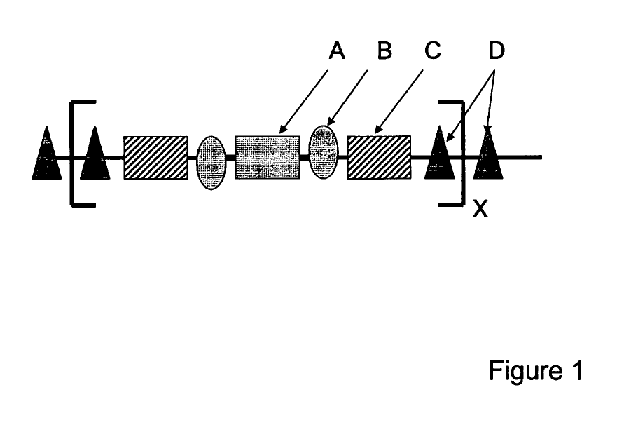

FIG. 2 illustrates an exemplary biodegradable elastomer in a branched degradable block co-polymer structure according to an embodiment of the present invention. A represents the pre-polymer block, B, the end group functionality, C, the degradable sequence, D, the bis-functionalized linker component and X is the degree of polymerization. The repeat unit is inscribed by the complex polygon.

FIG. 3 illustrates an exemplary biodegradable elastomer in a side chain protected pre-polymeric block structure providing for attachment sites for bioactive agents, such as growth factors, adhesion molecules or pharmaceutically active agents or prodrugs according to an embodiment of the present invention. A represents the end group used to incorporate the component into the block co-polymer, B, is the polymer chain, C, is the protected functionality (R) which may be the same as A or different, D, is the protecting group (PG), which can be removed for later attachment of other components.

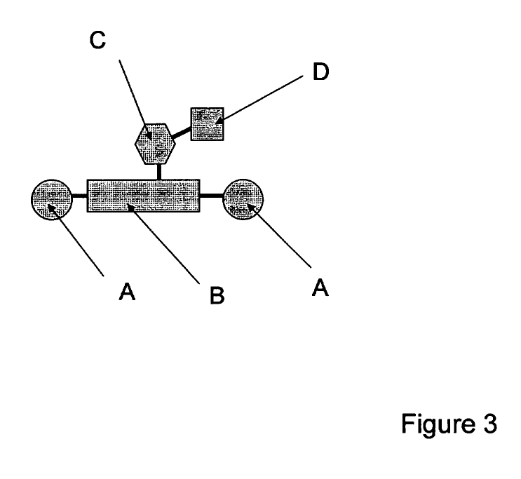

FIG. 4 illustrates the synthesis of an exemplary biodegradable elastomer, PEG-hydroxy acid biodegradable urethane elastomer, in a block copolymer structure according to an embodiment of the present invention. In this example, first the prepolymer is produced by adding Toluene to Sn(Oct)V at 100° C. for 24 hrs. Then the product is added to 4,4′-methylene-bis(dicyclohexyl-isocyanate), propylene glycol and a Sn(IV) catalyst at 140° C. for 4 hrs.

### DEFINITIONS

For the purposes of the present invention, the following terms shall have the following meanings:

For the purposes of the present invention, the term “biodegradable” refers to biodegradable elastomers that dissolve or degrade in vivo within a period of time that is acceptable in a particular therapeutic situation. Such dissolved or degraded product may include a smaller chemical species. Degradation can result, for example, by enzymatic, chemical and/or physical processes. Biodegradation takes typically less than five years and usually less than one year after exposure to a physiological pH and temperature, such as a pH ranging from 6 to 9 and a temperature ranging from 25° C. to 38° C.

For the purposes of the present invention, the term “biodegradable elastomer” refers to poly(urethanes) that include an ester linkage making them susceptible to degradation upon exposure to in-vivo conditions. These conditions may include physiological pH and temperature, for example, as well as physical contact with enzymes or other molecules present in-vivo. For purposes of this invention, the term “biodegradable elastomer” is interchangeable with the terms, “biodegradable poly(urethane), “poly(ester-urethane)”, “poly(urethane) with biodegradable ester linkages” and all singular or plural forms of the same.

For the purposes of the present invention, the term “combining” refers to any method of putting two or more materials together. Such methods include, but are not limited to, mixing, blending, commingling, concocting, homogenizing, incorporating, intermingling, fusing, joining, shuffling, stirring, coalescing, integrating, confounding, joining, uniting, and the like.

For the purposes of the present invention, the term “bioactive agent” refers to any agent with chemical or biological activity either in-vivo or in-vitro.

For the purposes of the present invention, ranges may be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Moreover, for the purposes of the present invention, the term “a” or “an” entity refers to one or more of that entity; for example, “a biodegradable elastomer” or “an bioactive agent” refers to one or more of those compounds or at least one compound. As such, the terms “a” or “an”, “one or more” and “at least one” can be used interchangeably herein. It is also to be noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated or biologically pure bioactive agent is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

This invention relates to biodegradable elastomers for use as medical implants and/or medical devices. The invention may further provide a method of delivering a bioactive agent through the use of such medical device.

According to a broad aspect of the invention, a new class of biodegradable elastomers is provided. Such elastomers are composed of poly(urethanes) with ester linkages to make them susceptible to degradation upon exposure to physiological conditions. Such biodegradable elastomers may be produced by any method known in the art. In a particular synthetic method, they are produced by condensing poly-ethylene glycol-b1-α-hydroxyacids with bis-isocyanates and short chain diols.

In one broad aspect of the present invention, biodegradable elastomers of the present invention are poly-α-hydroxyester urethanes.

Poly-α-hydroxyester urethanes may be obtained by reacting diisocyantes and polyols, such as PEG, with a cyclic lactone of an α-hydroxyester. Hydrolysis of the poly-α-hydroxyester-urethanes yields biocompatible by-products, such as lactic or glycolic acids, amines, alcohols, carbon dioxide and the like. The compositions of the present invention allow for alteration of the degradation rate by changing the molecular weight and chemical structure of the α-hydroxyacid blocks. The α-hydroxyester bonds may be readily cleaved under physiological conditions.

The invention may further provide polyvalent linkages built into the backbone of a biodegradable elastomer to allow for the addition of bioactive agents.

In another broad aspect of the present invention, the structure of the biodegradable elastomer includes a pre-polymeric block, a degradable sequence and a bis-functional linker.

The pre-polymeric block may include a polymeric or oligomeric diol or poly-ol. In one embodiment the pre-polymeric block is a diol and is selected from the group of polymers consisting of poly-propylene oxide, poly-ethylene oxide, poly(tetramethylene oxide), poly(trimethylene oxide) and the like. In a particular embodiment, the pre-polymeric block includes oligomers of propylene-oxide, ethylene oxide, tetramethylene oxide, trimethylene oxide and the like. In another embodiment, a diacid or poly acid may be used with similar poly ethers, such acids including but not limited to polymers selected from the group consisting of poly(-propyleneoxide), poly(-ethylene oxide), poly(tetramethylene oxide), poly(trimethylene oxide), and the like, terminated with carboxylic acids. The molecular weight of the pre-polymeric block or prepolymer, as well as its composition, can be varied depending on the characteristics desired in the biodegradable elastomer. One skilled in the art will understand how to utilize the pre-polymer block or prepolymer of interest in order to create the biodegradable elastomer of interest having a particular degradation rate.

The degradable sequences of the present invention may be any sequence degradable under physiological conditions. In one embodiment, they are attached to the ends of the pre-polymeric blocks. In another embodiment, the degradable sequence is conjugated to biodegradable linkages. These biodegradable linkages may be poly or oligo alpha-hydroxyesters or are composed of other degradable linkages, such as peptides, poly(orthoesters), poly(anhydrides), poly(acetals) or poly(ketals) or other degradable moieties known to those skilled in the art. Various types of degradable sequences may be found within a given biodegradable elastomer. The structure containing the central pre-polymeric block diol or poly-ol core or central diacid or poly-acid core can then be condensed into a polymeric structure through reaction with a bis-functionalized linker. The degradable sequence attached to the two ends of the pre-polymeric block may be the same or different. Additionally, they may be the same molecular weight or two different molecular weights. Variations may occur according to the various methodologies used to construct them.

Any bis-functionalized linker known in the art may be utilized with the present invention. In one embodiment, if the degradable sequences terminate in an alcohol, they can be reacted with bis-isocyanates to form urethanes, with bis-acid halides or activated bis-acids to form poly(esters) or with phosgene to form poly(carbonates). In another embodiment, if the degradable sequences terminate in an acid functional group they can be activated by chemical means and reacted with bis-amines to form poly amides, or with diols to form polyesters. In a third embodiment, if the degradable linkages terminate in amines they can be reacted with suitably activated diacids or diacid halides to form poly-amides. In a fourth embodiment, the linker component is a polymer. In a fifth embodiment the linker component has three or more reactive groups. For example it can be trifunctional or tetrafunctional or greater.

Any diisocyanates may be utilized for the preparation of the polyurethane of the block copolymers. In a particular embodiment, diisocyanates may be selected from the group consisting of hexamethylene diisocyanate, 2,2,4-trimethylhexamethylene diisocyanate, cyclohexyl-1,4-diisocyanate, cyclohexyl-1,2-diisocyanate, isophorone diisocyanate, methylenedicyclohexyl diisocyanate, L-lysine diisocyanate methyl ester and the like.

Any diacid halide may be used in the present invention. In a particular embodiment, the diacid halide is selected from the group consisting of the diacid halides of oxalic acid, malonic acid, succinic acid, glutaric acid, adipic acid, pimelic acid, suberic acid, azelaic acid, trimethyladipic acid, sebacic acid, dodecanedioic acid, tetradecanedioic acid, hexadecanedioic acid, and the like.

In another particular embodiment, diacids are selected from the group consisting of activated diacids of poly-propylene-oxide, poly-ethylene oxide, poly(tetramethylene oxide), poly(trimethylene oxide), and the like.

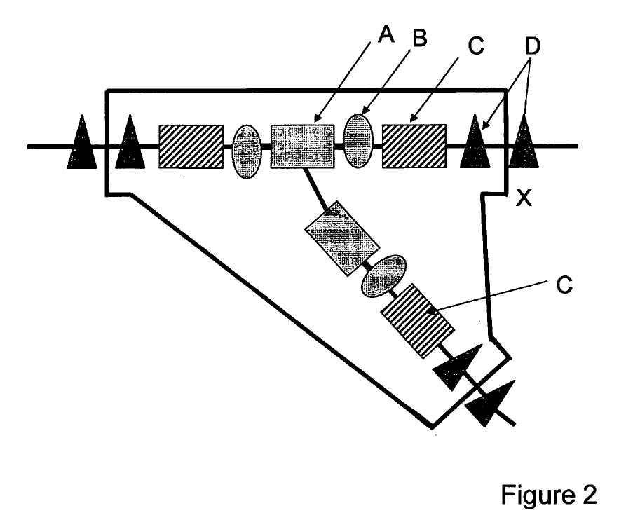

FIG. 2 illustrates an exemplary degradable elastomer with a structure including a branched degradable block copolymer with a plurality of sidechains. In this embodiment a biodegradable sequence could be attached to a multi-functional pre-polymeric block, which could then be condensed with a bis-functionalized linker. In this particular embodiment, A illustrates the pre-polymer block, B the end group functionality, C the degradable sequence and D the bis-functionalized linker. This unit may be repeated an unlimited number of times as required for a particular use.

The compositions of the present invention may further provide functionalized biodegradable elastomers. In one embodiment, the pre-polymeric block, degradable sequence and/or bis-functional linker is functionalized using a protected side chain attached to one or more of the three components. This side chain is then deprotected during the synthesis and bioactive agents may be attached. Such agents may be in native or prodrug form. The means to accomplish such attachment are well known to those skilled in the art.

The polymerization of the biodegradable elastomers can be of any degree. One skilled in the art knows how to manipulate polymerization in order to achieve the biodegradable elastomers of the present invention.

The polymers of the present invention may be of any structure. They may include a block, coblock or triblock configuration or any other structure that provides the degradation profile of interest to the end user. One skilled in the art, with the benefit of this disclosure, will be able to produce the biodegradable elastomer with the required properties by creating the appropriate structure.

In preparing the biodegradable elastomers of the present invention, the particular chemical, mechanical and degradation properties required for a particular use must be considered. One skilled in the art will understand how to produce biodegradable elastomers of the present invention with the properties of interest for a particular medical purpose with reference to the disclosure herein.

In another embodiment, the method of producing the biodegradable elastomer promotes the formation of crystalline and amorphous domains in the material.

Exemplary uses of the biodegradable elastomers according to the invention are described below. Further uses are of course possible.

The biodegradable elastomers of the present invention may be used in any medical device or implant.

In one embodiment, biodegradable elastomers are produced in tubular structures in solid, spiral-shaped, flexible, expandable, self-expanding, braided or tricot form, for example, which are sufficiently physically and pharmacologically structured or coated on the inside or outside according to the biological and functional requirements for the particular application.

If the biodegradable elastomers further provide bioactive agents, they may be held on the block copolymer by any method known in the art. In a particular embodiment, they are attached either by absorption, adsorption or by covalent chemical bonding. They may also be attached to any component of the biodegradable elastomer.

Other uses for the compositions of the present invention include stents for vessels or other biological tube structures, such as for the esophagus, biliary tract or urinary tract; and production of film-like structures for a variety of uses, such as wound covering, membrane oxygenators, cornea replacement base and the like.

Additionally, the compositions of the present invention may be made into thread-like structures, such as for surgical suture material or a base for woven, braided or tricot structures; or clip- or clamp-like structures for clamp suture apparatuses, clamps for ligature of small blood vessels and other devices requiring thermoplastic properties for closure. The present invention further may be used for the production of solid to gelatinous or porous structures as a matrix for the production of simple or composite biological tissues in vitro (tissue engineering) or in vivo, such as for preconditioned spacers for skin replacements, fatty tissue, tendons, cartilage and bone, nerves and the like. The compositions of the present invention may also be used for topical wound treatment.

In embodiments where the biodegradable elastomers are used as a drug-delivery device, such delivery may be through any route. The elastomers of the present invention may be formulated for parenteral administration, such as intravenous or intramuscular injection, but other alternative methods of administration may also be used, including, but not limited to, intradermal, pulmonary, buccal, transdermal and transmucosal administration. All such methods of administration are well known in the art.

Biodegradable elastomers may be produced such that they are capable of administration through a variety of routes merely by tailoring their biological charge properties and/or physical structures. For instance, such manipulations can be utilized to create foams, gels, microspheres, nanospheres, and the like. One skilled in the art is familiar with producing such biodegradable elastomers of the present invention demonstrating varying physical properties. The flexibility demonstrated by the present invention allows for release of a wide variety of agents, such as any therapeutic or cosmetic agent.

In a particular use, biodegradable elastomers of the present invention are of use for sclerosing varicoceles, varices of the legs, esophageal varices, or gastrointestinal sources of hemorrhage. In another particular embodiment, biodegradable elastomers of the present invention may be useful for the production of artificial auditory ossicles. Biodegradable elastomers may also be used as a base for the culture of corneal corpuscles on films for transplantation as cornea replacements.

Biodegradable elastomers may also be useful in the appropriate physical and/or biological form for medical or dental purposes. Additionally, they may be used in micro- or nanotechnology, or for a therapeutic and/or diagnostic imaging process, such as X-ray, CT, NMR, chemoembolization, PET or microscopy.

Bioactive agents of use in the present invention may be selected from the group consisting of, but not limited to, proteins, nucleic acids, carbohydrates, peptides, small molecule pharmaceutical substances, immunogens, metabolic precursors capable of promoting growth and survival of cells and tissues, antineoplastic agents, antihistamines, cardiovascular agents, anti-ulcer agents, bronchodilators, vasodilators, antiinfectives, anorexics, antiarthritics, antiasthmatics, anticonvulsants, antidepressants, antidiuretics, antidiarrheals, antiinflamatories, antibodies, radioactive agentc, cystostatics, hypnotics, muscle relaxants, sedatives, oligonucleotides, antigens, cholinergics, chemotherapeutics, analgesics, antihelmintics, antimigraine agents, central nervous system agents, narcotic antagonists and the like.

The bioactive agent may be in any form, such as a liquid, a finely divided solid or any other physical form. The biodegradable elastomers of the present invention may further include an additive selected from the group consisting of, but not limited to, diluents, carriers, excipients, stabilizers and the like.

The amount of bioactive agent will be dependent upon the particular agent employed and therapeutic purpose. The amount of drug can range from approximately 0.001% to about 70% of the biodegradable elastomer, from about 0.001% to about 50% of the biodegradable elastomer, to about 0.001% to about 20% of the biodegradable elastomer.

Biodegradable elastomers of the present invention may be produced by any method known in the art. In a particular embodiment, they are processed by extrusion or injection molding. Films of the present invention may be produced by any method known in the art. In a particular embodiment, they are produced by compression molding. Open-pored structures can be produced by various known processes, such as dipcoating or phase inversion or addition of salt to a solution of the block copolymer and precipitation of the polymer, for example.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Biodegradable Elastomers of Poly(Ester-Urethane)

In this example a biodegradable elastomer was produced by first reacting a polymeric diol (poly(tetrahydrofuran) or poly(tetramethylene glycol) with lactide to produce a biodegradable urethane. This reaction placed degradable units on both ends in a ABA type block copolymer. This polymer was then condensed with a bis-isocyanate to form the biodegradable urethane elastomer. This polymer is a biodegradable version of the well known medical grade polyurethane Tecoflex®.

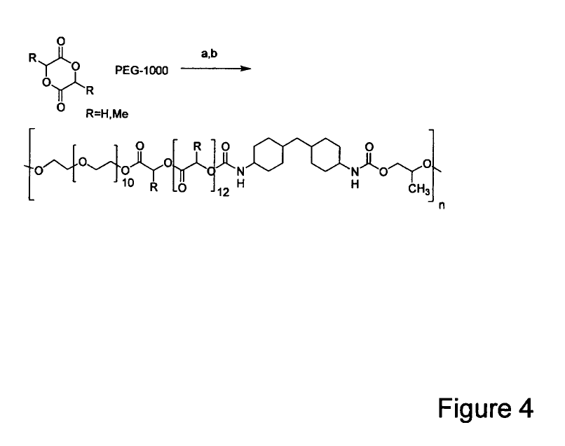

(a) Reaction of alpha, omega.-dihydroxy-(lactide)-poly(tetrahydrofuran)-(lactide) with 4,4′-Methylenebis(cyclohexylisocyanate) to produce the pre-polymer alpha, omega.-dihydroxy-(lactide)-poly(tetrahydrofuran)-PU (Reference FIG. 4):

Toluene (100 ml) was dried overnight over dehydrated molecular sieves (10 g). To a 500 ml flask, 6,6-dimethyl-1,4-dioxane-2,5-dione (20 g, 0.138 mol), 1000 MW poly(ethylene-glycol) (5.782 g, 0.00578 mol), tin (II) 2-ethylhexanoate, ˜90% in 2-ethyl-hexanoic acid (˜28% Sn) % Sn-28.29 (0.141 g, 0.141 mmol) was added prior to adding dried toluene (100 ml). A rubber septa capped reflux condenser was inserted into the top of the flask and a syringe, needle and balloon were inserted into the rubber septa such that excess pressure build-up in the mixture would be trapped in the balloon. Another needle connected to a Schlenk manifold was then inserted into the septa prior to flushing the apparatus with nitrogen three times. After confirming the reaction was under positive nitrogen pressure, the reaction was stirred and heated with a mantle and allowed to reflux for 24 hours using a soxlet extractor. After removal of the stir bar the flask was placed on ice for 20 minutes. The flask was then placed under a rotorary evaporator until 95% of the toluene was removed followed by high vacuum reduced pressure with slow stirring at 60° C. for 24 hours.

(b) Reaction of alpha, omega.-dihydroxy-(lactide)-poly(tetrahydrofuran)-(lactide) with 4,4′-Methylenebis(cyclohexyl isocyanate) to produce a biodegradable urethane elastomer:

The following procedure was run in triplicate. To a 25 ml scintillation vial was added the pre-polymer (7 g), prepared in (a) above. Tin (II)-2-ethylhexanoate (5.8 mg, 0.011 mmol) and ethylene glycol (0.466 g, 7.507 mmol) were added to the vial. After adding an airtight poly(ethylene) cap securely to the vial it was shaken using a KEM-Lab vortex mixer at 35 R.P.M. using one-second pulses for one minute. To the reopened vial, 4,4′-methylenebis(cyclohexyl isocyanate) (2.955 g, 11.262 mmol) was added. The mixture was capped and shaken by the vortex mixer for one minute. The vial was then placed into a heat-shaker for 15 minutes, removed and stirred with a stainless steel spatula for one minute in order to guarantee its consistency. The vial was recapped and the mixture was put back onto the shaker with the previous settings for 3.45 hours. Half of the hot mixture was then removed from the vial and placed into a second vial. 15 ml of N,N-dimethylacetamide (DMA) was added to each vial and the vials were put onto the shaker until the biodegradable elastomer was completely dissolved. To a 2 L flask the polymer/DMA mixture and approximately 1 L of water was added. The polymer/DMA mixture was stirred until the biodegradable elastomer was completely precipitated. The biodegradable elastomer was then removed from the water/DMA mixture and dried completely with towels. The dissolution/precipitation was repeated twice more. The biodegradable elastomer was then placed into a 200 ml flask, which was inserted into liquid nitrogen until it was frozen prior to being lyophilized overnight.

### Example 2

### Preparation of a Biodegradable Elastomer by Reaction of Alpha, omega.-dihydroxy-(lactide)-poly(ethylene glycol)-(lactide) with L-Lysine methyl ester diisocyanate

In this example a biodegradable urethane elastomer is produced by first reacting a polymeric diol (poly(ethylene glycol) with lactide. This reaction installed degradable units on both ends in a ABA type block copolymer. This polymer was then condensed with a bis-isocyanate (L-Lysine methyl ester diisocyanate) to form the degradable urethane elastomer. This type of biodegradable elastomer is composed of units that are either biological in origin (lysine and lactic acid) or of biocompatible polymers (poly(ethylene glycol).

(a) Synthesis of the Pre-Polymer: The pre-polymer materials utilized were 3,6-dimethyl 1,4 dioxane 2,5-dione (lactide) and PEG1000, both purchased through Sigma-Aldrich Corp (St. Louis, Mo.). The catalyst Tin Octanoate and Toluene were both purchased through EmSciences (Fort Washington, Pa.). The lactide, catalyst and PEG1000 were added to a round bottom flask in a 30:1:0.1 molar ratio respectively with 10 mL of toluene for every gram of lactide added. The toluene was then dried over-night with molecular sieve A4 (pore size 4 Å). A stir bar was added and the solution was refluxed over a mantle for 24 hours under positive nitrogen pressure. The pre-polymer was then stripped on a rotovap and allowed to dry over-night in a high-vac desiccator. The average molecular weight was found through maldi mass spectrometry.

(b) Polymer Synthesis: L-Lysine methyl ester diisocyanate (LDI) was purchased through Kyowa Hakko Kogyo Co. Ltd (Tokyo, Japan) and was purified by distillation before use. Dibutyltin dilaurate was obtained through Acros Organics (Geel, Belgium) and ethylene glycol was purchased from Fisher Chemical (Pittsburgh, Pa.). A study was done to assess the optimal equivalents and conditions for synthesis of the biodegradable urethane elastomer. The variables for the study were polymerization time (2, 4, 8 hours), temperature (90° C., 110° C.), LDI equivalents (2, 4, 6 equivalents), and catalyst concentration (0.05 g cat./g pre-polymer, 0.1 g cat/g pre-polymer). The study was done on a small scale in 5 ml vials. In each vial 0.1 grams of pre-polymer and varying equivalents of LDI and catalyst were added. The study was run at two temperatures 90° C. and 110° C. In all 24 vials were required to explore every possible combination. The biodegradable elastomers were then worked up by dissolving them in dimethyl acetamide (DMA) and precipitating the polymer through the addition of distilled water. This process was repeated 3 times and the biodegradable elastomer was then freeze dried by lyophilization. Rheometry was used to choose the biodegradable elastomer with the highest viscosity which was then selected and its reaction conditions were used for further study.

An accelerated degradation study was conducted by placing 30 samples in a bicine buffer solution at pH 8.5 and at 37° C. The samples were prepared by dissolving the biodegradable elastomer in tetrahydrofuran (THF) at a ratio of 1 mL of THF for every 0.17 grams of biodegradable elastomer. The solution was then divided into 4 salinized vials and allowed to dry in an oven. Samples were dried for 14 days with an air flow of 6 L/min and at a temperature of 55° C. The samples were then dried over night under high vacuum. The four films were each cut into 8 equal pieces, their weight was recorded, and each was placed in a 20 ml vial and 20 mL of bicine buffer solution was added. The vials were placed in a hot water bath set at 100 oscillations/min and 37° C. The study ran 10 days with 3 samples being taken off at 24 hour intervals. When each sample was removed from the water bath the remaining biodegradable elastomer was removed from the solution, placed in a tared vial, and stored in a freezer. Upon completion of the study the 30 tared vials were then freeze dried by lyophilization in order to find the dry mass of the remaining biodegradable elastomer. The buffer solution was refreshed halfway through the study.

### Example 3

### Preparation of a Biodegradable Urethane Elastomer by Reaction of alpha, omega.-dihydroxy-(lactide)-poly(ethylene glycol)-(lactide) with L-Lysine methyl ester diisocyanate

In this example a biodegradable urethane elastomer was produced by first reacting a polymeric diol (poly(ethylene glycol) of two different molecular weights with two equivalencies of lactide. This reaction installed degradable units on both ends in a ABA type block copolymer and made 4 different prepolymers. These polymer were then condensed with a bis-isocyanate (4,4′-methylenebis(cyclohexyl isocyanate) to form the biodegradable urethane elastomers. A series of studies were then conducted where the rates of degradation were measured as well as the mechanical properties indicating that by changing the structure a wide range of properties can be obtained.

(a) Synthesis of the block copolymer prepolymer: Six and 24 equivalents of 6,6-Dimethyl-1,4-dioxane-2,5-dione (Lac) were added to one equivalent of both 400 and 1000 Mw polyethylene glycol (PEG) to make four batches of pre-polymer as shown in Table 1 With respect to the PEG, 0.1 equivalent of tin (II) 2-ethylhexanoate, ˜90% in 2-ethyl-hexanoic acid (˜28% Sn) was distributed to each flask. 100 mL of dry toluene was added to each flask, and the mixtures were refluxed in a water free environment for 24 hours under positive nitrogen pressure using a Soxhlet extractor.

The tri-block copolymers were dried using a rotary evaporator for one hour and a liquid nitrogen desiccator for several days while stirring slowly at about 50° C. The pre-polymers were removed and MALDI mass spectrometry was used to confirm the average molecular weight of each sample.

TABLE 1
Variants of the triblock copolymer.

| | PEG | EQ of Lac | Units of Lac | Units of Lac |
|---|---|---|---|---|
| Polymer # - ID | MW | Added | Expected | Expected |
| 1 - Lac8PEG1000PU | 1000 | 24 | 12 | 8 |
| 2 - Lac2PEG1000PU | 1000 | 6 | 3 | 2 |
| 3 - Lac8PEG400PU | 400 | 24 | 12 | 8 |
| 4 - Lac2PEG4000PU | 400 | 6 | 3 | 2 |

(b) Synthesis of Biodegradable Polyurethane (PU) Elastomers. The viscous tri-block copolymers were each placed into tarred vials, and their estimated molar quantity was obtained. Three equivalents of 4,4′-methylenebis(cyclohexyl isocyanate) (MBCI), two equivalents of ethylene glycol and 0.0001 equivalents of tin (II)-2-ethylhexabiate were added to each vial. To each vial was then added two equivalents of ethylene glycol. A small stainless steel ball was placed into each vial before they were placed into a shaker and rapidly stirred for four hours. The “still hot” polymers were removed from the vials, placed into beakers and dissolved in N,N-dimethylacetamide (DMA) (˜15 mL per 5 grams of polymer). After the solutions appeared homogenous, distilled water (DIH₂O) was continually added to precipitate the polymer and to carefully wash out the DMA. Low molecular weight components were washed out as well. The dissolution/re-precipitation process was repeated twice more. The final products were drained of water, frozen in liquid N₂ to −72° C. and lyophilized.

Two quantities of each product were placed into separate vials. Seven extra equivalents of lactide were added to one vial and they were both run through the said polymerization process.

TABLE 2
Three measurements of molecular weight, Z average (Mz), weight
average (Mw), number average (Mn), and polydispersity (Pd)
(Pd = Mw/Mn) for three PUs. The averages were taken from
three samples. The relative standard deviation (RSD) is the
SD/Average * 100%.

| | | | Number | |
|---|---|---|---|---|
| | Z-average | Weight | Average | Polydispersity |
| Lac₂PEG₁₀₀₀PU | (M_{z}) | Average (M_{w}) | (Mₙ) | (Pd) |
| Average | 68,209 | 25,350 | 3,152 | 8.042 |
| SD | 3232 | 440 | 45 | 0.185 |
| RSD | 4.74% | 1.74% | 1.43% | 2.30% |
| Lac₂PEG₁₀₀₀PU | M_{z} | M_{w} | Mₙ | Pd |
| Average | 18,370 | 9,195 | 1,245 | 7.442 |
| SD | 1408 | 115 | 133 | 0.862 |
| RSD | 7.66% | 1.25% | 10.64% | 11.58% |
| Lac₂PEG₄₀₀PU | M_{z} | M_{w} | Mₙ | Pd |
| Average | 146,491 | 51,798 | 3,895 | 13.314 |
| SD | 5007 | 549 | 178 | 0.529 |
| RSD | 3.42% | 1.06% | 4.57% | 3.97% |

Characterization (stress/strain): They were then placed into large vials where they could be dissolved in tetrahydrofuran (THF) as a 0.05 g/mL (polymer/THF) composition. After they were dissolved, the tight caps were removed, and two mL of each mixture was placed into round wells of a flat bottom Teflon® block. The block was then placed into a closed oven where a constant 2 L/min air-flow could be passed over the top allowing efficient evaporation of the THF. After several days the biodegradable elastomer films were removed from the block. A hammer and a fabricated 4 mm wide steel stamp were used to cut the films into small dog-bone shapes approximately a quarter-diameter in length. Five dog-bones of each polymer were tested on a load-cell for six parameters designated by the American Society for Testing and Materials (ASTM) (yield strain, yield strength, failure strain, failure strength, breaking toughness, and Young's modulus), which were collected for statistical analysis across the four polymers. This analysis was done via a two tailed t-test comparing each quality between each of the six possible combinations of the four polymers. Yield strain, yield strength, failure strain, failure strength, breaking toughness and Young's modulus are shown for the four polymers in Table 3. These parameters all initially appeared to be relatively large for Lac₈PEG₁₀₀₀PU. Lac₂PEG1000PU initially appeared to have the second largest parameters followed by Lac₂PEG₄₀₀PU and then Lac₈PEG₄₀₀PU. Standard deviations across these sets of measurements were somewhat large. Two-tailed t-test analysis revealed that, when comparing any two of the four PUs against one another, the Young's modulus was not different with probability value (P-value) less than 0.05.

TABLE 3
Young's modulus (E), yield strain (YS), yield strength (YSG), failure
strain (YS), failure strength (FSG) and breaking toughness (B.T.)
were calculated for each of the four polymer sets. LacnPEG1000PU
consistently gave the higher stress and strain values.

| Parameter | Lac₈PEG₁₀₀₀PU | Lac₂PEG₁₀₀₀PU |
|---|---|---|
| E (MPa) | 158.87 ± 13.91 | 144.40 ± 47.37 |
| YS | 0.14 ± 0.03 | 0.13 ± 0.02 |
| YSG (MPa) | 12.71 ± 1.26 | 12.33 ± 1.70 |
| FS | 1.47 ± 0.12 | 1.47 ± 0.27 |
| FSG (MPa) | 17.18 ± 1.33 | 16.66 ± 1.80 |
| B.T. (MPa) | 20.48 ± 1.06 | 19.42 ± 3.87 |
| Parameter | Lac₈PEG₄₀₀PU | Lac₂PEG₄₀₀PU |
| E (MPa) | 113.40 ± 27.43 | 121.06 ± 11.10 |
| YS | 0.13 ± 0.03 | 0.13 ± 0.01 |
| YSG (MPa) | 8.34 ± 1.40 | 9.50 ± 0.30 |
| FS | 0.88 ± 0.07 | 1.40 ± 0.19 |
| FSG (MPa) | 10.35 ± 0.91 | 11.55 ± 0.56 |
| B.T. (MPa) | 8.04 ± 0.64 | 13.71 ± 2.06 |

(b) Characterization (degradation study): Each of the four polymers was dissolved in THF for 24 hours so that there was a 0.16 g/mL (polymer/THF) composition. Approximately 17 mL of each homogeneous mixture was placed into 25 mL silane coated scintillation vials. The vials were then placed into an oven, at room temperature, with 2 L/min of air flowing across the open tops. Two weeks later the thick polymer films were removed from the vials and checked to insure that they were completely dry by examining negligible mass loss over the course of 24 hours. The polymer that lined the walls of the vial was trimmed off leaving quarter-shaped discs. Three circles of each polymer were cut into eight approximately equal pieces summing to 24 pieces for each polymer.

The pieces were weighed and placed into clean scintillation vials. Each polymer containing vial was charged with approximately 20 mL of 0.043 M, pH 7.4, phosphate buffered saline (PBS), capped, and placed into a water bath/shaker where they were rotated 107 rpm at 37° C.

Each week three vials from each polymer batch (12 vials total) were removed from the bath. The pieces were rinsed several times with DIH₂O to remove any excess degraded product, which also insured precision in the weighing process. The pieces were placed into tarred vials, which were immersed in liquid nitrogen and hence frozen to −72° C. The frozen contents were then lyophilized, and the vials with corresponding dried polymer pieces were weighed again. The mass of the vial was subtracted from the final mass of the vial/dried polymer revealing the final mass of the pieces. The relative degradation rates as functions of Lac content and PEG length for the four polymers were compared against one another. After 120 days Lac2PEG400PU degraded 10%, Lac8PEG400PU, degraded 40%, Lac2PEG1000PU, degraded 40%, Lac8PEG1000PU, degraded 50%. Two PEG1000 polymers show the effect of bulk vs. surface degradation as the lower Lac allows impregnation of the polymer with water molecules. The PEG400 polymers on the other hand are both hydrophobic and both surface degrade. This difference in available degradable units causes a difference in degradation.

All of the compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions, and methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

1. A biodegradable elastomer comprising:
(a) a pre-polymeric block;
(b) a degradable sequence; and
(c) a bis-functionalized linker.

2. The biodegradable elastomer of claim 1, wherein said pre-polymeric block is selected from the group consisting of a polymeric diol, an oligomeric diol, a polymeric poly-ol, an oligomeric poly-ol, a diacid, a polyacid, a multifunctionalized polyether, an alpha omega.-dihydroxypoly(ethylene oxide), an alpha omega-dihydroxypoly(propylene oxide), an alpha omega-dihydroxypoly(ethylene oxide co-propylene oxide), an alpha omega-dihydroxypoly(tetramethylene oxide), an alpha omega-dihydroxypoly(butylene oxide), a multifunctionalized polyester, a poly(caprolactone), a poly(butyrolactone), a poly(orthoesters) and their block copolymers.

3. The biodegradable elastomer of claim 1, wherein said degradable sequence is selected from the group consisting of poly(phosphazenes), poly(orthoesters), poly(anhydrides), poly(acetals), oligo(phosphazenes), oligo(orthoesters), oligo(anhydrides), and oligo(acetals).

4. The biodegradable elastomer of claim 1, wherein said degradable sequence is connected to said biodegradable elastomer in a manner selected from the group consisting of an attachment to the ends of the pre-polymeric block, condensation to said pre-polymeric block and conjugated to biodegradable linkages.

5. The biodegradable elastomer of claim 3, wherein said degradable sequence is an alpha-omega-dihydroxypolyester obtained by ring-opening polymerization of cyclic esters, lactones or mixtures thereof in the presence of an aliphatic diol, polyetherdiol or polyesterdiol.

6. The biodegradable elastomer of claim 1, wherein said degradable sequence is susceptible to degradation upon exposure to physiological conditions.

7. The biodegradable elastomer of claim 1, wherein said bis-functionalized linker is selected from the group consisting of a bis-isocyanate, a bis isothiocyanate, a bis-N-hydroxysuccinimidyl ester, a bis-acid halide, a bis epoxide, a bis halide, bis-alkyne, a bis-alkene, a phosgene, an activated bis-acid, a bis-amine, a diol, a diacid, a diacid halide and a bis-acid azide.

8. The biodegradable elastomer of claim 1, further including a bioactive agent.

9. The biodegradable elastomer of claim 8, wherein said bioactive agent is selected from the group consisting of proteins, nucleic acids, carbohydrates, peptides, small molecule pharmaceutical substances, immunogens, metabolic precursors capable of promoting growth and survival of cells and tissues, antineoplastic agents, antihistamines, cardiovascular agents, anti-ulcer agents, bronchodilators, vasodilators, antiinfectives, anorexics, antiarthritics, antiasthmatics, anticonvulsants, antidepressants, antidiuretics, antidiarrheals, antiinflamatories, antibodies, radioactive agentc, cystostatics, hypnotics, muscle relaxants, sedatives, oligonucleotides, antigens, cholinergics, chemotherapeutics, analgesics, antihelmintics, antimigraine agents, central nervous system agents and narcotic antagonists.

10. The biodegradable elastomer of claim 1, wherein said biodegradable elastomer is a component of a medical device.

11. The biodegradable elastomer of claim 10, wherein said biodegradable elastomer is in the form of a tube with one or more channels.

12. The biodegradable elastomer of claim 10, wherein said biodegradable elastomer has a porous structure.

13. The biodegradable elastomer of claim 10, wherein said biodegradable elastomer is biocompatible.

14. The biodegradable elastomer of claim 1, wherein the degradation rate of said biodegradable elastomer is altered by varying the properties in said pre-polymeric block, degradable sequence and bis-functionalized linker of said biodegradable elastomer.

15. The biodegradable elastomer of claim 14, wherein said properties are selected from the group consisting of molecular weight and chemical structure.

16. (canceled)

17. (canceled)

18. A biodegradable elastomer comprising: poly-alpha-hydroxyester urethane susceptible to degradation upon exposure to physiological conditions.

19. A biodegradable elastomer comprising: the reaction product of diisocyanate, a polyol and an alpha-hydroxyester, wherein said biodegradable elastomer is susceptible to degradation upon exposure to physiological conditions.

20. A method of providing a medical device comprising:
a. a pre-polymeric block;
b. a degradable sequence;
c. a bis-functionalized linker, and
d. a bioactive agent; and exposing said device to physiological conditions by implanting said device in a patient, wherein said bioactive agent is released by exposure to physiological conditions.

21. The method of claim 20, wherein said pre-polymeric block is selected from the group consisting of a polymeric diol, an oligomeric diol, a polymeric poly-ol, an oligomeric poly-ol, a diacid, a polyacid, a multifunctionalized polyether, an alpha omega.-dihydroxypoly(ethylene oxide), an alpha omega-dihydroxypoly(propylene oxide), an alpha omega-dihydroxypoly(ethylene oxide co-propylene oxide), an alpha omega-dihydroxypoly(tetramethylene oxide), an alpha omega-dihydroxypoly(butylene oxide), a multifunctionalized polyester, a poly(caprolactone), a poly(butyrolactone), a poly(orthoesters) and their block copolymers.

22. The method of claim 20, wherein said degradable sequence is selected from the group consisting of poly(phosphazenes), poly(orthoesters), poly(anhydrides), poly(acetals), oligo(phosphazenes), oligo(orthoesters), oligo(anhydrides), and oligo(acetals).

23. The method of claim 20, wherein said degradable sequence is connected to said biodegradable elastomer in a manner selected from the group consisting of an attachment to the ends of the pre-polymeric block, condensation to said pre-polymeric block and conjugated to biodegradable linkages.

24. The method of claim 22, wherein said degradable sequence is an alpha-omega-dihydroxypolyester obtained by ring-opening polymerization of cyclic esters, lactones or mixtures thereof in the presence of an aliphatic diol, polyetherdiol or polyesterdiol.

25. The method of claim 20, wherein said degradable sequence is susceptible to degradation upon exposure to physiological conditions.

26. The biodegradable elastomer of claim 20, wherein said bis-functionalized linker is selected from the group consisting of a bis-isocyanate, a bis isothiocyanate, a bis-N-hydroxysuccinimidyl ester, a bis-acid halide, a bis epoxide, a bis halide, bis-alkyne, a bis-alkene, a phosgene, an activated bis-acid, a bis-amine, a diol, a diacid, a diacid halide and a bis-acid azide.

27. The method of claim 20, wherein said bioactive agent is selected from the group consisting of proteins, nucleic acids, carbohydrates, peptides, small molecule pharmaceutical substances, immunogens, metabolic precursors capable of promoting growth and survival of cells and tissues, antineoplastic agents, antihistamines, cardiovascular agents, anti-ulcer agents, bronchodilators, vasodilators, antiinfectives, anorexics, antiarthritics, antiasthmatics, anticonvulsants, antidepressants, antidiuretics, antidiarrheals, antiinflamatories, antibodies, radioactive agentc, cystostatics, hypnotics, muscle relaxants, sedatives, oligonucleotides, antigens, cholinergics, chemotherapeutics, analgesics, antihelmintics, antimigraine agents, central nervous system agents and narcotic antagonists.

28. A composition comprising; the reaction product of poly-ethylene glycol-b1-alpha-hydroxyacid, a bis-isocyanate and a short chain diol, said reaction product having the characteristic of degrading upon exposure to physiological conditions.

29. A material for a medical device comprising;
a. a pre-polymeric block;
b. a degradable sequence; and
c. a bis-functionalized linker, wherein said material is susceptible to degradation upon exposure to physiological conditions.

30. The material of claim 29, wherein said degradation rate is altered by varying the properties in said pre-polymeric block, degradable sequence and bis-functionalized linker of said material.

31. The material of claim 29, further providing a bioactive agent.
